(19) United States
(12) Patent Application Publication
(10) Pub. No.: US 2011/0034692 A1
(43) Pub. Date: Feb. 10, 2011

(54) SPECIFIC IMPURITIES OF MONTELUKAST

(75) Inventors: Ales Halama, Pardubice (CZ); Olga Bouskova, Praha (CZ); Petr Gibala, Praha (CZ); Josef Jirman, Praha (CZ)

Correspondence Address: Pearl Cohen Zedek Latzer, LLP, 1500 Broadway, 12th Floor, New York, NY, 10036, US

(73) Assignee: ZENTIVA, K.S., Praha 10 (CZ)

(21) Appl. No.: 12/922,267

(22) PCT Filed: Mar. 11, 2009
(86) PCT No.: PCT/CZ2009/000038

§ 371 Date: Oct. 25, 2010

(30) Foreign Application Priority Data
Mar. 14, 2008 (CZ) PV 2008-167

(51) Int. Cl. C07D 215/18

(52) U.S. Cl. 546/174

(57) ABSTRACT

The subject-matter of the invention consists in a method of removing specific impurities of montelukast of formula (I), which occur due to chemical instability of the target substance and also contaminate the substance in the preparation process. Further, methods of isolation of specific impurities of montelukast defined by formulae (V-A), (IV-A), (XIIIa-A), (XIIIb-A) and analytic methods used for the control of the production of montelukast in the pharmaceutical quality.

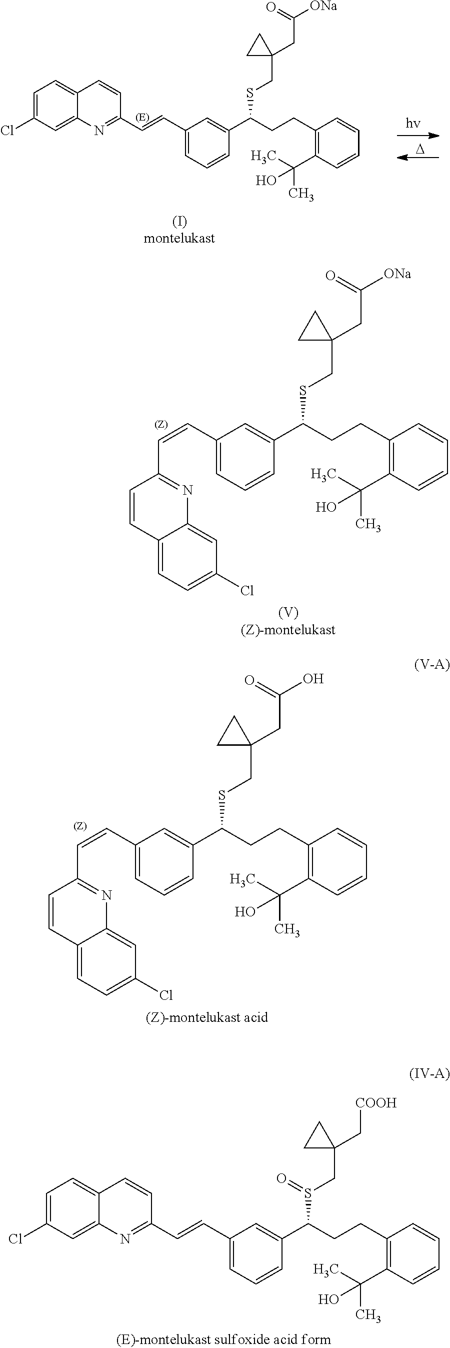

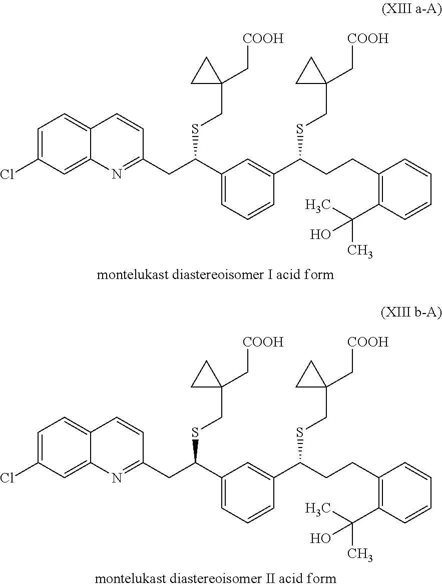

### TECHNICAL FIELD

The present invention deals with a new method of obtaining chemically pure and pharmaceutically acceptable montelukast sodium (I), or a method of removing specific impurities that are generated either due to the intrinsic instability of montelukast or are produced in the process of its preparation.

### BACKGROUND ART

Montelukast sodium (I) is an active ingredient of products used for the treatment of respiration diseases, mainly asthma and nasal allergy. Montelukast sodium, chemically the sodium salt of [R-(E)]-1-[[[1-[3-[2-(7-chloro-2-quinolinyl)ethenyl]phenyl]-3-[2-(1-hydroxy-1-methylethyl)phenyl]propyl]thio]-methyl]cyclopropane acetic acid is described by the chemical formula (I).

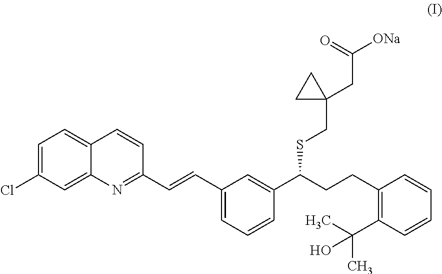

The first solution of chemical synthesis of montelukast (I) was described in the patent no. EP 0480717 B1 and subsequently in specialized literature as well (M. Labele, Bioorg. Med. Chem. Lett. 5 (3), 283-288 (1995)). More possibilities of chemical synthesis of montelukast (I) are described in the following patents: EP 0480717 B1, EP 0737186 B1, US 2005/0234241 A1, WO 2005/105751 A1, US 2005/0107612 A1, WO 2005/105749 A2, WO 2005/105750 A1, US 2007/208178 A1.

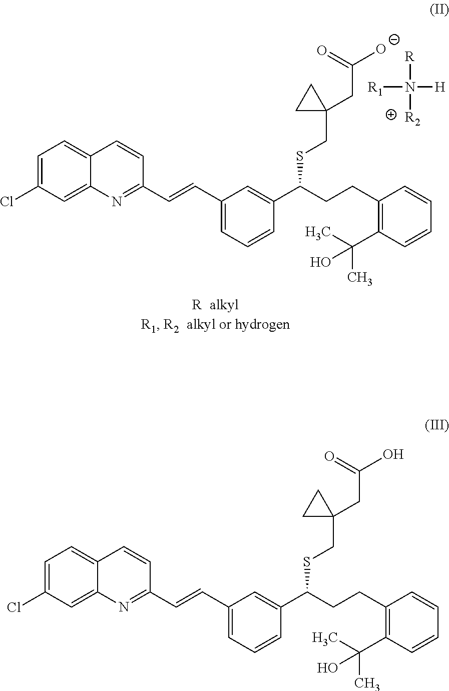

For the process of isolation and purification of crude montelukast salts of montelukast with some amines (II) or montelukast acid (III) in the solid state have been used so far. Among montelukast salts with amines salts with dicyclohexylamine (EP 0737186 B1, WO 04108679A1), tert-butylamine (US 2005/0107612 A1, WO 06043846A1), ethylphenylamine (US 2005/0107612 A1), isopropylamine (WO 2007/005965 A1), di-n-propylamine (WO 2007/005965 A1) and with cycloalkylamines (C5-C9, US 2007/213365 A1) have been described. Solid forms of montelukast acid, both crystalline and amorphous, have been described in a number of patent applications: WO 2005/040123, WO 2005/073194 A2, WO 2005/074893 A1, WO 2005/074893 A1, WO 2004/108679 A1, WO 2005/074935A1. The most common method used in practice consists in purifying crude montelukast (I) via its salts with secondary amines, mainly with dicyclohexylamine (EP 0737186 B1).

The sodium salt of montelukast, its preparation and various forms, amorphous or crystalline, are described in a number of patents or patent applications, e.g. amorphous montelukast sodium is dealt with by EP 0737186 B1, WO 03/066598 A1, WO 2004/108679 A1, WO 2005/074893 A1, WO 2006/054317A1 a WO 2007/005965. Crystalline polymorphs of montelukast sodium are described by WO 2004/091618 A1 and WO 2005/075427 A2.

Processes of isolation and purification of montelukast are of crucial economic significance as they make it possible to obtain a substance that can be used for pharmaceutical purposes. These processes are used to remove impurities that result from the chemical instability of montelukast as well as the instability of the raw materials used for its chemical synthesis or non-selectivity of chemical reactions, or they may be represented by residues of the raw materials used, especially solvents. There is a general rule that chemical purity of the active pharmaceutical ingredient (API) produced in the industrial scale is one of the critical parameters for its commercialization. The American Food and Drug Administration (FDA) as well as European medicament control offices require, according to the Q7A ICH (International Conference on Harmonization) instruction, that API is freed from impurities to the maximum possible extent. The reason is achieving maximum safety of using the drug in the clinical practice. National inspection and control offices usually require that the content of an individual impurity in an API should not exceed the limit of 0.1%. All the substances (generally referred to as impurities) contained in an API over the limit of 0.1% should be isolated and characterized in accordance with the ICH recommendations. It is also recommended to isolate and characterize degradation products that are generated during the storage or usability period of API (ICH Guideline, 2006). In order to obtain information about the stability of a substance and to describe degradation products so-called “stress tests” are performed. Within these tests the API is subjected to a series of critical conditions the selection of which depends on the structure of the tested API. Usually, the influence of an increased temperature, air humidity, light, oxygen and stability in a wide pH range is assessed.

Before being used in a pharmaceutical product every API must be analyzed for chemical purity. For this purpose usually High Performance Liquid Chromatography, HPLC, is used. Impurities present in the API are then determined by the relative position of the peak in the HPLC chromatogram while the peak position is typically expressed as time (in minutes) necessary for the impurity to get from the place of injection of the sample into the HPLC column filled with a suitable absorbent up to the detection place. The time that is necessary, under standard conditions, for the chemical (e.g. an API or impurity) to travel from the injection place to the detector is referred to as the “retention time”. Retention times (rt) related to the standard retention time (usually rt of the API) are called “relative retention times”. The relative retention time (rrt) of the API typically has the value 1; the constituents that get to the detector in a shorter time manifest retention times lower than 1 while the constituents that travel more slowly show relative retention times higher than 1. Under standard conditions the relative retention times are considered as constant characteristics of the analyzed substance, i.e. they only depend on the chemical structure of the corresponding substance.

The position of the peak in the chromatogram, or the retention time is only a quality parameter that does not provide information about the quantity of the analyzed substance. But the area under every peak that belongs to the respective constituent is proportional to the concentration of the analyzed constituent. The determined content of a constituent in a sample is typically expressed in %. The content of the constituent in percent is calculated from the value of the area under the peak of the constituent divided by the sum of the areas under all the peaks in the chromatogram and the result subsequently multiplied by 100. The sum of the contents of all the constituents, including the API, then equals the value of 100%. For unambiguous determination of the retention times of the analyzed substances it is necessary to obtain standards of both the API alone and the individual impurities. For the standards it is first necessary to verify correctness of the chemical structure with suitable methods. For this purpose spectral methods are typically used, especially NMR (Nuclear Magnetic Resonance), MS (Mass Spectroscopy), or a combination of a separation and spectral techniques, e.g. LC-MS (combination of liquid chromatography and mass spectroscopy). As soon as the chemical structures of the API standards as well as of the isolated impurities, an analytic method can be developed (e.g. HPLC) that will allow assessing the impurity of each produced API batch in a standard and reproducible way. Isolated impurities can be used in HPLC as “external” or “internal” standards. For the purposes of quantity determination impurity standards are used in the “standard addition” method or for the determination of “response factors” (Strobel H. A., Heineman W. R., Chemical Instrumentation: A Systematic Approach (Wiley & Sons: New York 1989), Snyder L. R., Kirkland J. J. Introduction to Modern Liquid Chromatography (John Wiley & Sons: New York 1979)). If standards of impurities are not available, it is very difficult to determine their actual content in the API, to find an acceptable analytic method and to validate it. Without the possibility of reliable assessment of the quality of API its production process cannot be controlled and the obtained substance cannot be used for the preparation of a pharmaceutical product. The standards of impurities and the methods of analyzing chemical purity of the API have the crucial importance for the control of the production process and subsequently for successful commercialization of the product.

In the montelukast molecule there are a number of functional groups that impair the chemical stability of this substance. Montelukast is known to be prone to several types of degradation; it is mainly the case of three kinds of chemical transformation:
(a) Oxidation of the mercapto group to the sulphoxide according to equation (1),

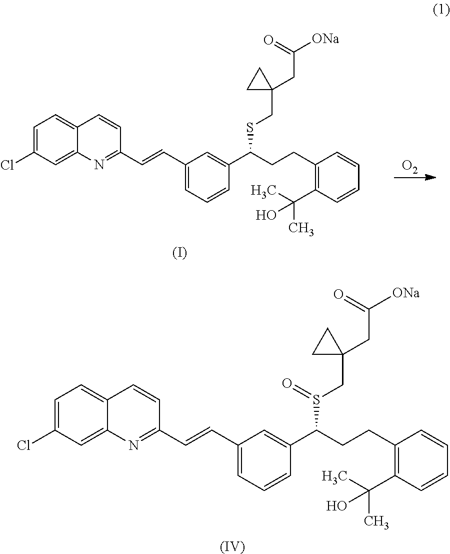

(b) Isomerisation at the location of the double bond from geometry (E) to (Z), or trans to cis by the effect of light according to equation (2),

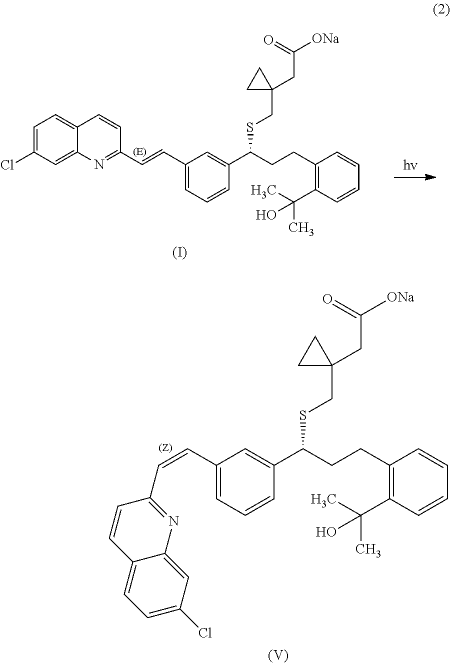

(c) Dehydration at the location of tert. alcohol, producing the corresponding olefin according to equation (3).

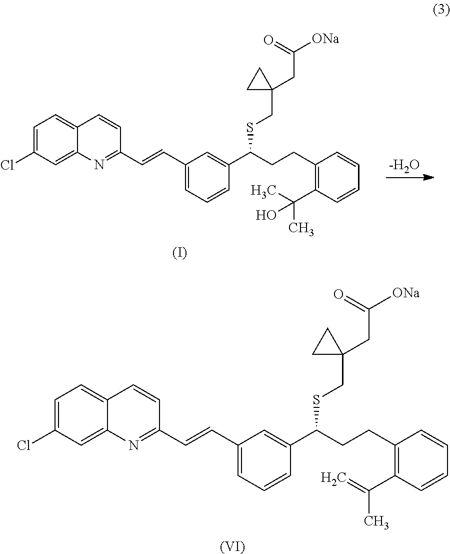

Literature (E. D. Nelson, J. Pharm. Sci. 95, 1527-1539 (2006), C. Dufresne, J. Org. Chem. 1996, 61(24), 8518-8525, WO 2007005965A1) describes increased sensitivity of montelukast (or rather the mercapto group, which montelukast contains) to oxygen, see equation (1)). As the main product of oxidation of montelukast (I) (E)-montelukast-sulfoxide, chemically the sodium salt of [R-(E)]]-1-[[[1-[3-[2-(7-chloro-2-quinolinyl)ethenyl]phenyl]-3-[2-(1-hydroxy-1-methylethyl)-phenyl]propyl]sulfinyl]methyl]cyclopropane acetic acid, described with chemical formula (IV), is mentioned. Contamination of the product with this impurity is undesirable. For this reason the processes leading to the target substance are carried out with the exclusion of oxygen, i.e. under the protective atmosphere of an inert gas (e.g. nitrogen according to EP 0737186 B1). (E)-Montelukast-sulfoxide (IV) has also been described as a product of the oxidative metabolism of montelukast (Balani S. K. et al: Drug Metabolism and Disposition (1997) 25 (11), 1282-87, Dufrense C.: J. Org. Chem. (1996) 61(24), 8518-25).

Exposure of montelukast to light causes its isomerization while a montelukast derivative with geometry (Z) is generated in the location of the double bond (Smith Glen A. et al: Pharm. Res. 2004, 21(9), 1539-44). The impurity resulting from photo-instability is (Z)-montelukast, chemically the sodium salt of 1-[[[(1R)-1-[3-[(1Z)-2-(7-chloro-2-quinolinyl)ethenyl]phenyl]-3-[2-(1-hydroxy-1-methylethyl)phenyl]propyl]thio]methyl]cyclopropane acetic acid, which is described by chemical formula (V), see equation (2).

Another degradation impurity described in literature (WO 2007005965A1) is montelukast dehydrated, chemically the sodium salt of 1-[[[(1R)-1-[3-[(1E)-2-(7-chloro-2-quinolinyl)ethenyl]-phenyl]-3-[2-(1-methylethenyl)phenyl]propyl]thio]methyl]cyclopropane acetic acid, described by chemical formula (VI), see equation (3).

The organic impurities of the target substance have their origin in chemical instability of montelukast as well as instability of the ingredients used for its synthesis or these may be residues of the used raw materials or solvents. An example of a source of contamination due to instability of intermediate products is the commonly used ingredient montelukast mesylate, chemically 2-(2-(3(S)-(3-(2-(7-chloro-2-quinolinyl)ethenyl)phenyl)-3-methanesulfonyl-oxypropyl)phenyl)-2-propanol, characterized by formula (VII). Montelukast mesylate is prepared via a reaction of the relatively stable montelukast alcohol, chemically 2-(2-(3(S)-(3-(2-(7-chloro-2-quinolinyl)ethenyl)-phenyl)-3-methanesulfonyloxypropyl)-phenyl)-2-propanol, characterized by formula (VIII), and methane sulfonyl chloride. Subsequently montelukast mesylate is converted by the action of a salt of [1-(mercapto-methyl)cyclopropyl]acetic acid with an alkaline metal (IX) to the target montelukast, see Scheme 1. In parallel to this reaction the considerably instable montelukast mesylate (VII) is subject to undesired intramolecular conversions (J. O. Egekeze, Anal. Chem. 1995, 67, 2292-2295) described by Scheme 1. Via an elimination reaction the impurity montelukast eliminate, chemically 2-[2-(3-{3-[2-(7-chloroquinolin-2-yl)vinyl]phenyl}allyl)phenyl]-propan-2-ol, described by formula (X), is generated from the intermediate (VII). A cyclization reaction produces another impurity, namely montelukast cyclizate, chemically 7-chloro-2-{2-[3-(1,1-dimethyl-1,3,4,5-tetrahydrobenzo[c]-oxepin-3-yl)phenyl]vinyl}quinoline, described by formula (XI), from the intermediate (VII).

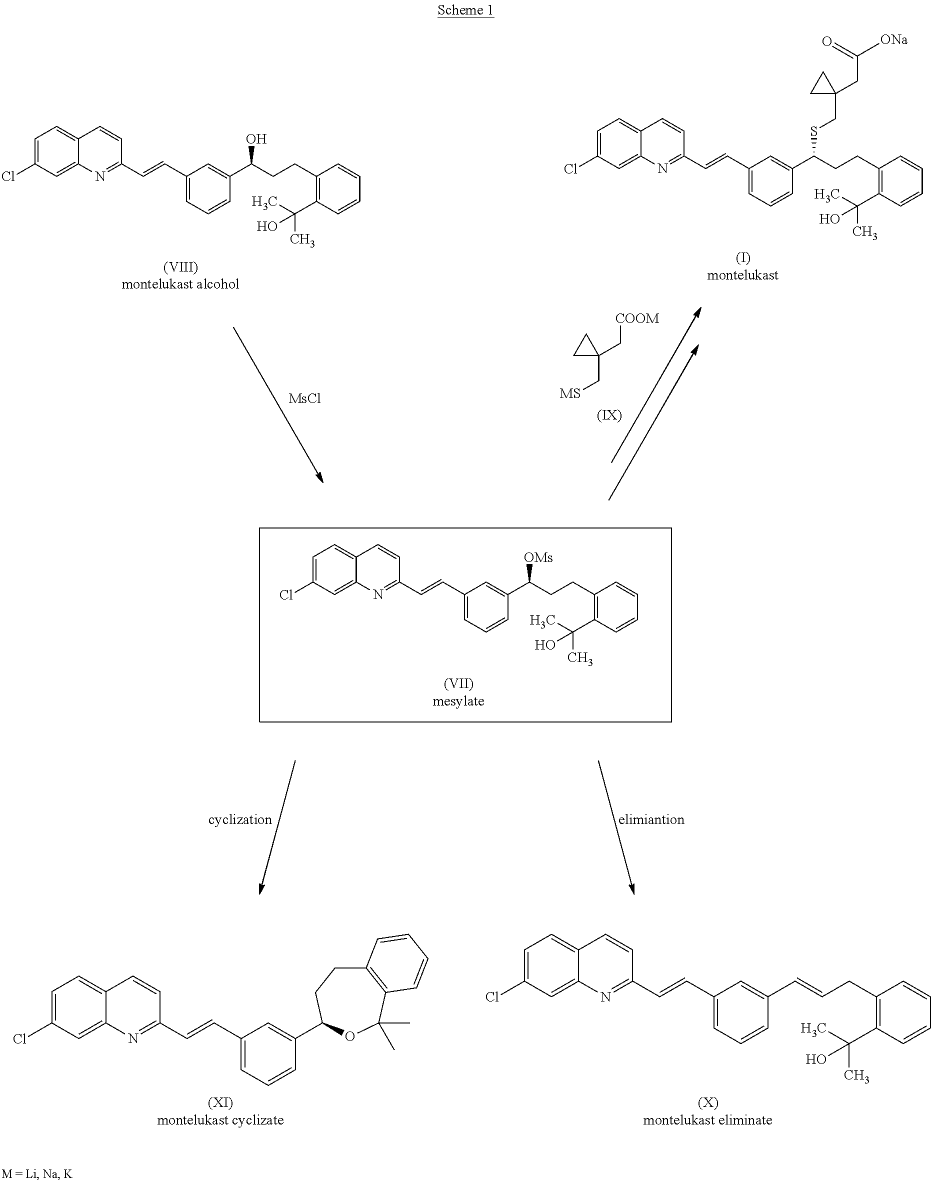

The considerable chemical instability of montelukast and its intermediates also influences its industrial production. The preparation processes of montelukast sodium are usually based on prevention of the formation of impurities, mainly those that result from photo-instability and oxidation instability. This goal can be achieved by carrying out the production in equipments that are impermeable for light and working under an inert atmosphere with the exclusion of air oxygen. Chemical impurities of montelukast are usually removed by means of crystallization in the phase of its salts with amines or in the phase of montelukast acid. The target form of the API is the sodium salt of montelukast, which cannot be efficiently further purified by common procedures since the resulting substance is soluble very well in numerous solvents from polar ones (e.g. water, ethanol) to non-polar ones (e.g. diethyl ether, toluene). An exception is represented by non-polar solvents of the heptane, hexane, pentane and cyclohexane type.

Our solution represents a new and beneficial way of obtaining pure montelukast sodium (I) with simultaneous removal of specific impurities below accepted limits.

### DISCLOSURE OF INVENTION

The invention consists mainly in processes concerning carrying out and controlling the chemical purification of montelukast for the purpose of removing specific impurities. Specific impurities are generated due to chemical instability of the target substance, which results from the structure of the target substance, or the substance gets contaminated during the preparation process, which can be attributed to non-selectivity of the chemical processes in the preparation of montelukast. Other objects of the invention include methods of isolation of specific impurities of montelukast and analytic methods used for controlling the production process and the final quality of montelukast.

The reactions leading to the target compound (I) were performed, according to the process now discovered, in such a way that first [1-(mercaptomethyl)cyclopropyl]acetic acid was mixed with sodium tert-butylate and polyethylene glycol (PEG) in the environment of an inert organic solvent and under the atmosphere of an inert gas. The obtained mixture was cooled below −5° C. and then a solution of 2-(2-(3(S)-(3-(2-(7-chloro-2-quinolinyl)ethenyl)phenyl)-3-methanesulfonyloxypropyl)phenyl)-2-propanol (VII) was added dropwise. Further, the reaction mixture was maintained under the inert atmosphere and stirred for several hours. Samples were continuously taken to determine the conversion and selectivity of the reaction. Crude montelukast sodium was then converted to a solution of montelukast acid (III) and further isolated and purified in the form of crystalline salts of montelukast with primary amines (II). The target amorphous form of montelukast sodium was obtained by direct conversion of the salt of montelukast with the primary amine by action of sodium tert-butylate as a suitable source of sodium ions. The process used is described in a detailed way in Examples 1 to 5.

Stress tests were used to assess factors influencing the stability of the obtained montelukast, mainly its sensitivity to light and oxygen. For the purpose of analyzing the chemical stability a methanolic solution of montelukast was exposed to the influence of sunshine and air oxygen while this solution was continuously analyzed by means of the HPLC method (Example 6). The found high sensitivity of the substance to light and air oxygen is documented by FIG. 2, which shows chromatograms of the methanolic solution of montelukast exposed to the influence of sunshine and air oxygen at various times from the beginning of degradation of the starting substance. It has been found out that the transformation of montelukast (I) to (Z)-montelukast (V) was unexpectedly fast. Already within minutes the concentration of (Z)-montelukast may grow to a level of units of percent. Simultaneously, but more slowly, the mercapto group was oxidized, producing (E)-montelukast sulfoxide (IV). Besides the well-known impurities formation of another, not yet described substance, which was a product of both the chemical processes, isomerism on the double bond as well as oxidation of the mercapto group, was observed. The substance was (Z)-montelukast sulfoxide, chemically the sodium salt of [R-(Z)]]-1-[[[1-[3-[2-(7-chloro-2-quinolinyl)ethenyl]phenyl]-3-[2-(1-hydroxy-1-methylethyl)-phenyl]propyl]sulfinyl]methyl]cyclopropane acetic acid, described by chemical formula (XII); see Scheme 2.

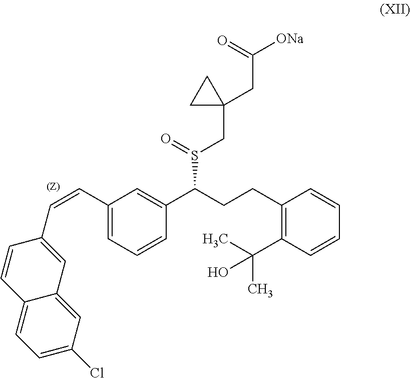

(Z)-Montelukast sulfoxide (XII) is exactly the final product in the whole sequence of undesired reactions induced by light or oxygen, which take place according to Scheme 2. This compound is a product of the second generation of degradation transformations of montelukast. With regard to the relatively slow transformation of montelukast to (Z)-montelukast-sulfoxide (XII) this degradation impurity does not belong to critical impurities, unlike the degradation impurities of the first generation (e.g. (IV) and (V)). With regard to the rate of undesired degradation changes the most critical impurity is the (Z) isomer of montelukast (V).

The impurities generated by degradation of the target substance are, on one hand, structurally very similar to the target substance and therefore it is very difficult to reduce their content in the API by common methods (e.g. crystallization). On the other hand, they are generated relatively easily and so they can contaminate the substance that had been subjected to the purification process and was already found acceptable in terms of content of impurities. For this reason it is advantageous to dispose with methods of removing impurities at the very end of the production process as well, i.e. suitable methods of reprocessing a substance that was contaminated by undesired impurities e.g. in the course of drying, storage or transport.

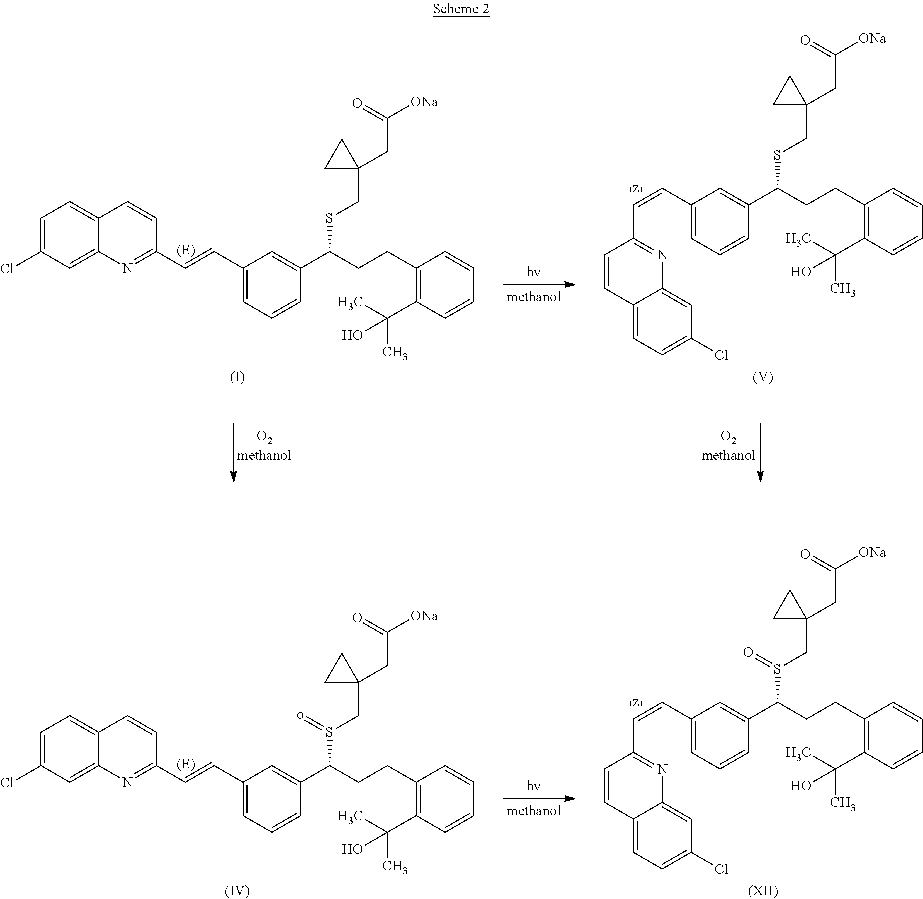

An interesting, unexpected and preferably process-feasible effect has been found for montelukast and its (Z)-isomer. While exposure to light induces an increase of the content of (Z)-montelukast (V), in the case of heat exposure exactly the opposite is true; see equation (4). A montelukast solution maintained under the atmosphere of inert argon was first exposed to sunshine for a defined time period, while the concentration of the (Z)-isomer according to HPLC grew from the value of the original content below 0.1% to the value of 11% (Example 7). This mixture of the two isomers was boiled in a light-insulated apparatus under decrease of the content of the (Z)-isomer in the mixture. When this back transformation of the undesired (Z)-isomer to the desired (E) isomer was implemented in a toluene solution, then the half-time of this reaction was 30 minutes; see FIG. 1. The chemical transformation of (Z)-montelukast to montelukast, induced by heat exposure of a solution of a mixture of the two substances represents a simple and advantageous way of reprocessing specifically contaminated montelukast sodium (I). An important aspect of the inventive process for the removal of the (Z)-isomer of montelukast from the target substance involves carrying out the purification operation directly in the final form of the API (the sodium salt), without the necessity of converting the API to another, well-crystallizing form.

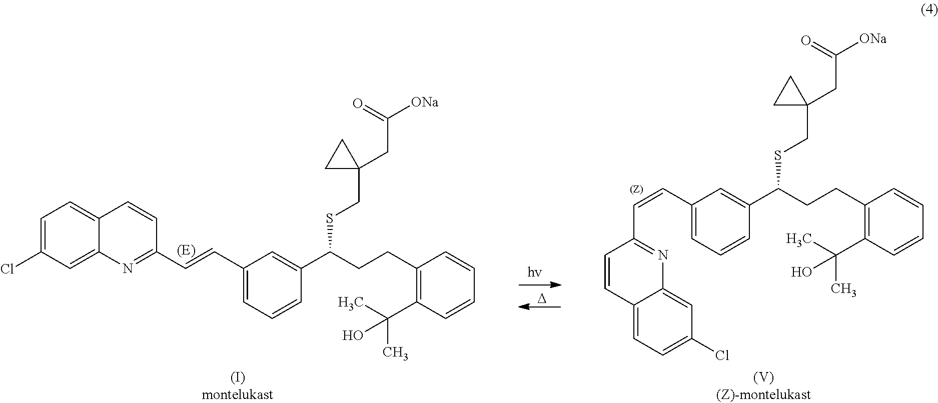

Literature has not yet described a method for reprocessing of montelukast in case of later contamination of the API by degradation products or other impurities. However, its quality may be deteriorated very easily, e.g. during drying of the API, when the substance is exposed to an increased temperature, or during storage and transport. According to the inventive process (see Scheme 3) contaminated montelukast can be efficiently reprocessed by transformation to a well-crystallizing form, e.g. to a salt of montelukast with an amine. Montelukast sodium contaminated with a specific impurity is dissolved in a suitable solvent, it is first transformed to a solution of montelukast acid (III) by the action of a solution of an acid and then to the well-crystallizing salt (II) by the action of an amine (RR₁R₂N). Further, it is necessary to remove impurities by crystallizations of the isolated salt of montelukast with the amine (II) from a suitable solvent or more solvents. The selection of a suitable solvent depends on the type of the impurity removed. If montelukast is contaminated with polar specific impurities, polar solvents can be preferably used, e.g. alcohols, ketones, esters or nitriles. If montelukast is contaminated with non-polar specific impurities, non-polar solvents can be preferably used, e.g. ethers, chlorinated hydrocarbons or aromatic hydrocarbons. After the removal of specific impurities the salt of montelukast with the amine (II) is transformed to the target sodium salt of montelukast. The yields comprising both isolation and crystallization of the salt of montelukast with amines and transformation of these salts to the sodium salt of montelukast are about 75%; the achieved chemical purity was higher than 99.5% (HPLC) with the contents of individual impurities below 0.1% (Example 8). The inventive process, which is described in Scheme 3, can be used for reprocessing montelukast (I) of poor quality to a pharmaceutically acceptable API.

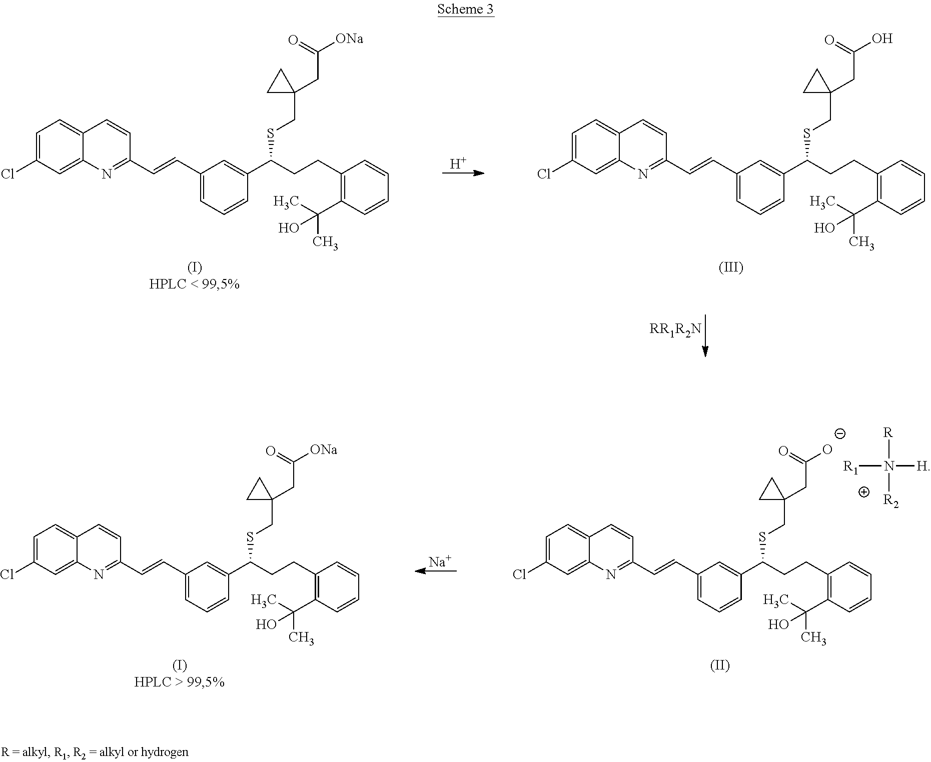

Besides the impurities that come from the decomposition of the target substance the API usually contains also specific impurities that have their origin in the production process. Impurities of this type differ from the degradation impurities mainly by the fact that their content in the target substance does not grow any further. In crude montelukast sodium (I) a mixture of diastereoisomers of the sodium salts of 2-[(R)-1-[[[1-[3-[2-(7-chloro-2-quinolinyl)-(S)-1-({[1-(carboxy-methyl)cyclopropyl]-methyl}thio)ethyl]phenyl]-3-[2-(1-hydroxy-1-methylethyl)phenyl]propyl]-thio]methyl]-cyclopropane]acetic acid and 2-[(R)-1-[[[1-[3-[2-(7-chloro-2-quinolinyl)-(R)-1-({[1-(carboxymethyl)cyclopropyl]methyl}thio)ethyl]phenyl]-3-[2-(1-hydroxy-1-methylethyl)-phenyl]propyl]thio]methyl]-cyclopropane]acetic acid, defined by formulas (XIII a) and (XIII b) has been found as a specific and so far not known impurity. These substances are generated through a sequence of reactions of alkali metal salts of [1-(mercaptomethyl)-cyclopropyl]acetic acid (IX) with the very reactive and chemically instable 2-(2-(3 (S)-(3-(2-(7-chloro-2-quinolinyl)ethenyl)phenyl)-3-methanesulfonyloxypropyl)-phenyl)-2-propanol (VII); see Scheme 4. The impurities (XIII a) and (XIII b) are thus specific for montelukast sodium (I) prepared by processes using, as the reagent, a salt of [1-(mercaptomethyl)-cyclopropyl]acetic acid with alkali metals (IX) selected from the group of lithium, sodium and potassium.

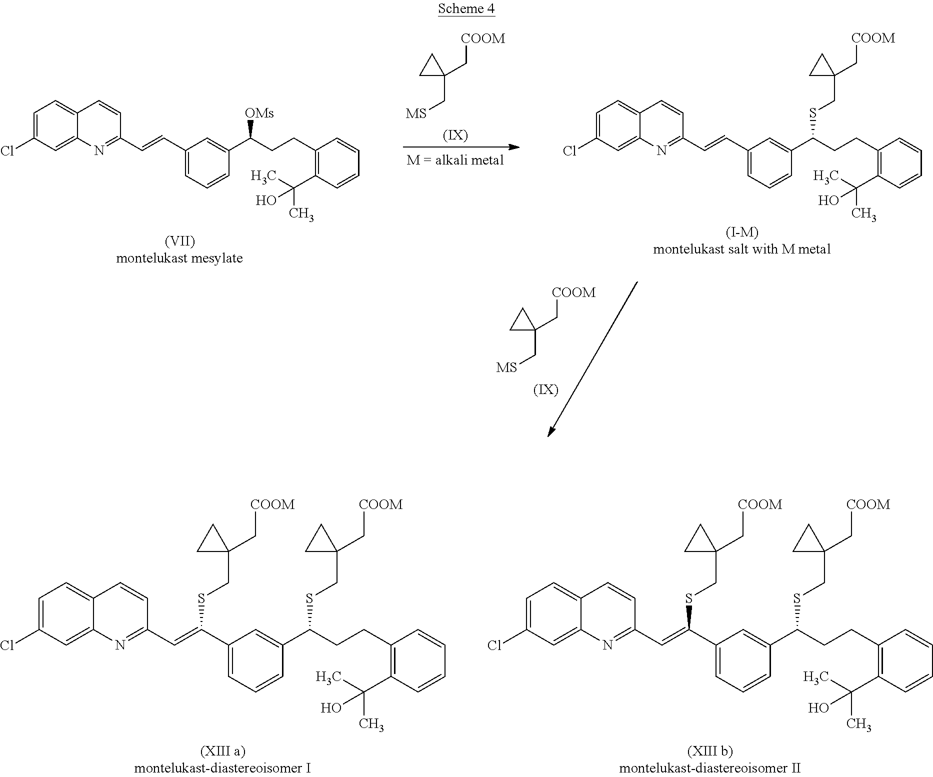

The contents of diastereoisomers (XIII a) and (XIII b), referred to as montelukast diastereoisomer I and montelukast diastereoisomer II in a simplified manner, are being successfully removed in the process of chemical synthesis of montelukast by means of crystallization of salts of montelukast with amines in polar solvents. For this purpose salts of montelukast with primary amines are suitable, especially with isopropylamine and n-propylamine. As suitable polar solvents alcohols, ketones, esters or nitriles can be used, e.g. isopropyl alcohol, acetone, ethyl acetate and acetonitrile. The process of reduction of the contents of the diastereoisomers (XIII a) and (XIII b) is described in a more detailed way in Example 8.

Instability of montelukast mesylate (VII), i.e. the starting compound typically used in the chemical synthesis of montelukast (I), may be the source of even more impurities of the target substance. In particular, it is the case of montelukast cyclizate (XI) and montelukast eliminate (X). Both the degradation products of montelukast mesylate (VII) are being successfully removed in crystallizations of salts of montelukast with amines (II), especially from non-polar solvents. Due to insufficient conversion the target substance may even be contaminated with montelukast alcohol (VIII), which is the starting compound for the preparation of montelukast mesylate (VII). Montelukast alcohol (VIII) is being successfully removed in crystallizations of salts of montelukast with amines, especially from polar solvents.

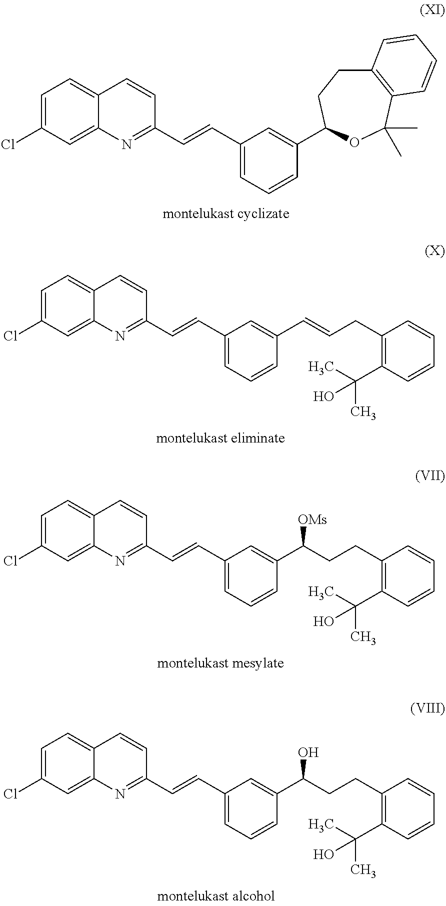

For the development of analytic methods it was necessary to obtain standards of specific impurities of montelukast, namely (Z)-montelukast (V), (E)-montelukast sulfoxide (IV), and diastereoisomers (XIII a) and (XIII b). For this purpose, these standard do not necessarily have to be in the form of the sodium or another salt; other acido-basic forms are equally usable, e.g. free acids that are structurally described by the chemical formulae (V-A), (IV-A) (XIII a-A) and (XIII b-A). The specific impurities (V), (IV), (XIII a) and (XIII b), or their free acids (V-A), (IV-A) (XIII a-A) and (XIII b-A) are characterized by their mutual structural similarity as well as structural similarity to montelukast, which makes their isolation more difficult. Therefore, for the preparation of standards separation methods were conveniently used, mainly the Waters auto-purification system.

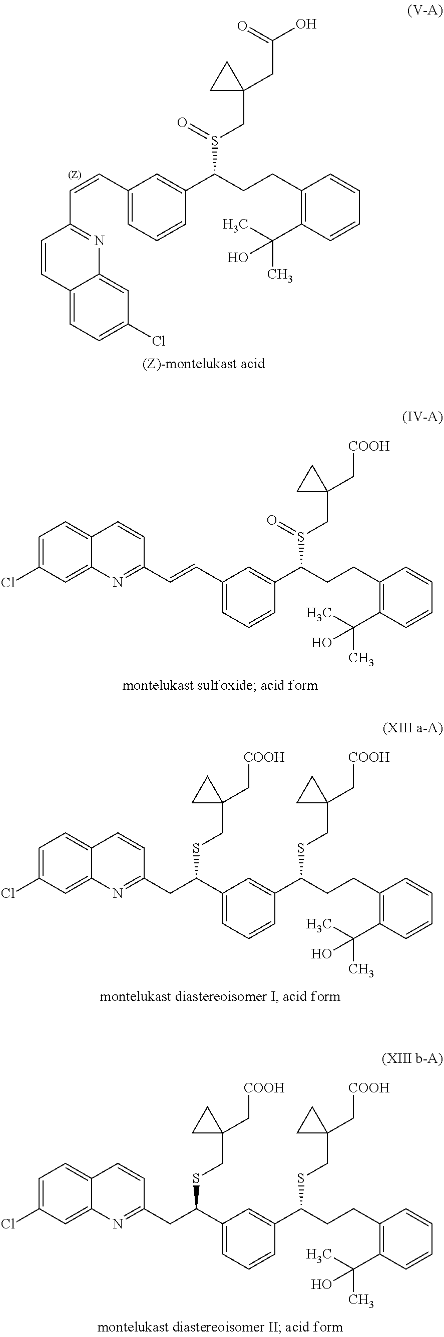

The Waters auto-purification system is a combination of various chromatographic instruments integrated in a specific configuration that enables automated purification or isolation of particular substances from a sample on the basis of a signal from a UV and MS detector. The Waters auto-purification system comprises and analytic column, which is used for optimization of the separation and verification of purity of collected fractions, and also a semi-preparative column for the entire separation of larger volumes and concentrations of injected samples. Injection of samples and collection of fractions is controlled by the sample manager. Collection of fractions is carried out on the basis of signal intensity from the UV or MS detector exceeding the preset threshold value. Signals from the UV and MS detector can also be combined with the use of logical operators, which allows a high purity of collected fractions to be achieved.

The standards of the specific impurities were obtained by separation from mixtures in which the concentration of the required impurity was increased in a targeted way. Thus, for example, by exposure of a methanolic solution of montelukast to light a mixture of substances was obtained where the (Z)-isomer of montelukast predominated. Subsequent separations resulted in separation of other constituents and in obtaining the standard of (Z)-montelukast (V), or (Z)-montelukast acid (V-A). The standard of (E)-montelukast sulfoxide (IV) was obtained by separations of the crude product obtained from oxidative degradation of montelukast performed with the use of hydrogen peroxide. Separations from concentrated mother liquors obtained during the preparation of montelukast provided the standards of both the diastereoisomers (XIII a-A) and (XIII b-A).

The standard of dehydrated montelukast (VI) was prepared by acid catalyzed dehydration of montelukast under the condition of azeotropic distillation with toluene. The preparation of the standard (VI) is described in a more detailed way in Example 11. The dehydration product was not detected at all in the target substance prepared by the process we used (according to Examples 1 to 5); in spite of this fact the standard (VI) was used for optimum setting of the analytic method of controlling the chemical purity of the API (HPLC with gradient elution).

The standards of both the degradation products of montelukast mesylate (VII), montelukast cyclizate (XI) and montelukast eliminate (X) were successfully isolated from a mixture obtained after heat loading of montelukast mesylate (VII) by the process according to Example 13.

The structure of all the prepared standards was verified by means of spectral methods (NMR and MS). The preparation procedures of standards of montelukast impurities, including the methods of their separation and isolation, are described in a more detailed way in Examples 9 to 13.

Analytic methods of quality control, which have to be sufficiently reliable and precise, are an integral part of every API production process. For the control of the preparation process and of the quality of the target montelukast (I) it was necessary to develop analytic methods that will be able to distinguish both previously known and newly found inventive impurities of montelukast. For this purpose two methods of high performance liquid chromatography (HPLC) have been developed. The method working in the isocratic mode was mainly designed to control the composition of reaction mixture, while the method working in the gradient mode was mainly designed to assess the quality of the target product and isolated intermediates. Both the methods have the advantage of easy and quick performance and, in the case of the gradient method, also excellent distinction of all possible impurities, including the input ingredients and intermediates. Both chromatographic methods are described in a more detailed way in the experimental part.

The present invention concerns an advantageous and efficient method of removing specific chemical impurities of montelukast (I), which can contaminate the substance designed for the preparation of a drug for treatment of asthma and allergies. The benefits of the inventive process consist in isolation of specific impurities, by means of which the analytic methods that can be conveniently used for the quality control of montelukast have been optimized. A very significant aspect of the present solution is represented by processes allowing re-processing of montelukast contaminated by products of its degradation. The used processed of re-processing of contaminated montelukast differ according to the type of the specific impurity. A very advantageous process has been found for the removal of the (Z)-isomer of montelukast (V) by heat exposure of a solution containing a mixture of montelukast and its (Z)-isomer. The other degradation impurities can then be removed by a process using well-crystallizing salts of montelukast with amines (II). The inventive purification processes, methods of chemical analysis and standards of specific impurities can be very preferably used for the production of montelukast sodium in the quality required for pharmaceutical substances.

### BRIEF DESCRIPTION OF DRAWINGS

FIG. 1. HPLC obtained by isocratic elution of a solution of a mixture of montelukast isomers (Z)/(E) boiled in toluene without accession of light (according to Example 7).

Sequence of peaks: 1—(Z)-montelukast (V), 2—montelukast (I).
(a) Chromatogram of the initial mixture of isomers obtained according to Example 7 (content of (Z)-montelukast 11%)
(b) Chromatogram of the sample taken from the mixture after 30 minutes of reflux (content of (Z)-montelukast 5.6%)
(c) Chromatogram of the sample taken from the mixture after 60 minutes of reflux (content of (Z)-montelukast 2.7%)
(d) Chromatogram of isolated montelukast (content of (Z)-montelukast below 0.1%)

FIG. 2. HPLC chromatograms obtained by isocratic elution of a methanolic solution of montelukast exposed to the influence of sunshine and air oxygen.

Sequence of peaks: 1—(Z)-montelukast sulfoxide (XII), 2—(E)-montelukast sulfoxide (IV), 3—(Z)-montelukast (V), 4—montelukast (I).
Samples of the methanolic solution analyzed at times:
(a) 40 minutes, (b) 1 day, (c) 4 days, (d) 14 days

FIG. 3. HPLC chromatogram obtained by gradient elution of a montelukast solution with the additions of standards of specific impurities. The content of each added impurity is 10% with regard to montelukast.

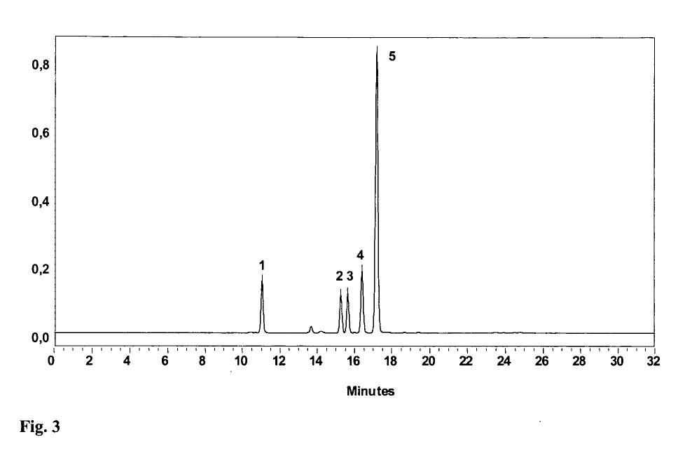

Sequence of peaks: 1—(E)-montelukast sulfoxide (IV), 2—montelukast diastereoisomer I (XIII a), 3—montelukast diastereoisomer II (XIII b), 4—(Z)-montelukast (V), 5—montelukast (I)

FIG. 4. HPLC chromatogram obtained by gradient elution of a montelukast solution with the additions of standards of formerly known as well as newly found impurities.

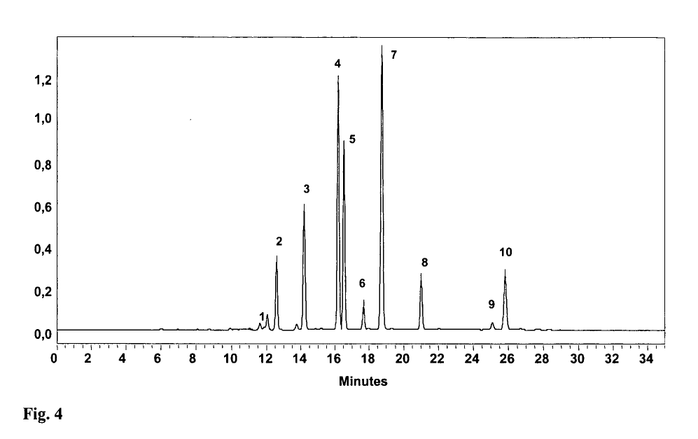

Sequence of peaks: 1—(Z)-montelukast sulfoxide (XII), 2—(E)-montelukast sulfoxide (IV), 3—montelukast alcohol (VIII), 4—montelukast diastereoisomer I (XIII a), 5—montelukast diastereoisomer II (XIII b), 6—(Z)-montelukast (V), 7—montelukast (I), 8—montelukast eliminate (X), 9—montelukast dehydrated (VI), 10—montelukast cyclizate (XI)

### EXAMPLES

The object of the invention will be explained in a more detailed way in the following examples, which, however, have no influence on the scope of the invention defined in the claims.

### Example 1

### Synthesis, Crude Montelukast Sodium

In 200 ml of toluene [1-(mercaptomethyl)cyclopropyl]acetic acid (6.62 g), a base (sodium tert-butoxide, 8.50 g) and PEG-600 (26 ml in 30 ml of toluene) were mixed together, the mixture was stirred under argon and cooled to ca. −10° C. To the obtained slurry a solution of 2-(3-(S)-(3-(2-(7-chloroquinolinyl)-ethenyl)phenyl)-3-methanesulfonyloxypropyl)phenyl-2-propanol (26 g) in 120 ml of tetrahydrofuran was subsequently added. The reaction mixture was gradually stirred at from −10° C. to the laboratory temperature for 1 hour. The stirring was continued at the laboratory temperature for a number of hours. The reaction mixture was continuously analyzed by means of HPLC (isocratic mode). At the end of monitoring the reaction mixture contained 85.7% of montelukast.

### Example 2

### Isolation of the Salt of Montelukast with iso-propylamine

The reaction mixture of Example 1 was concentrated in vacuum, 100 ml of toluene were added to the residue and concentrated in vacuum again. The residue was diluted with toluene to the volume of 200 ml. It was washed twice with 0.5 M solution of tartaric acid, twice with 100 ml of water and the obtained toluene solution was dried over sodium sulfate. Then, the desiccant was filtered off and 50 ml of acetonitrile, 4.5 ml of iso-propylamine and 200 ml of heptane were added. After one hour of stirring another 100 ml of heptane were added to the suspension and the stirring continued for one hour. Then, filtration was performed and the cake was washed with 3×50 ml of heptane. After vacuum drying at the laboratory temperature 19.7 g of an off-white powder were obtained. The yield, comprising both the synthesis of the crude sodium salt of montelukast according to Example 1 and isolation of the salt with iso-propylamine was 75%; HPLC 93.5%.

The salt of montelukast with n-propylamine was obtained in an analogous way. The yield comprising both the synthesis of the crude sodium salt of montelukast and isolation of the salt with n-propylamine was 68%; HPLC 94.3%.

### Example 3

### Crystallization of the Salt of Montelukast with iso-propylamine

15.0 g of the salt of montelukast with iso-propylamine were mixed with 200 ml of toluene and, under argon atmosphere, stirred and gradually heated up to 95° C. Then, under intensive stirring the mixture was slowly cooled down to the laboratory temperature and further stirred for several hours. Then, it was filtered and the cake was washed with 2×50 ml of heptane. After vacuum drying at the laboratory temperature 12.9 of an off-white powder were obtained. Crystallization yield 86%; HPLC 99.7%.

¹H NMR (250 MHz, DMSO-D6), δ (ppm) 0.23-0.47 (m, 4H, 2×CH₂ cyclopropyl), 1.08 (d, 6H, 2×CH₃ iso-propyl), 1.44 (s, 6H, 2×CH₃), 2.10-2.30 (m, 4H, 2×CH₂), 2.51 (m, 1H, CH), 2.52 and 2.63 (m, 2H, CH₂), 2.77 and 3.07 (2×m, 2H, CH₂), 3.06 (m, 1H, CH iso-propyl), 4.01 (t, 1H, CH), 5.70 (bb, 4H, NH³⁺, OH), 7.03-8.41 (m, 15H, CH═CH and CH-arom.).

The salt of montelukast with iso-propylamine was crystallized in an analogous way from acetonitrile (1 g dissolved under boiling in 40 ml of solvent, yield 65%) from acetone (1 g dissolved under boiling in 10 ml of solvent, yield 46%) from ethyl acetate (1 g dissolved under boiling in 40 ml of solvent, yield 67%) from ethanol (1 g dissolved at the temperature of 55° C. in 10 ml of solvent, yield 45%) from isopropyl alcohol (1 g dissolved at the temperature of 55° C. in 10 ml of solvent, yield 70%).

### Example 4

### Crystallization of the Salt of Montelukast with n-propylamine

15.0 g of the salt of montelukast with n-propylamine were mixed with 200 ml of toluene and, under argon atmosphere, stirred and gradually heated up to 95° C. Then, under intensive stirring the mixture was slowly cooled down to the laboratory temperature and further stirred for several hours. Then, it was filtered and the cake was washed with 2×50 ml of heptane. After vacuum drying at the laboratory temperature 11.7 g of an off-white powder were obtained. Crystallization yield 78%; HPLC 99.7%.

¹H NMR (250 MHz, DMSO-D6), δ (ppm) 0.25-0.45 (m, 4H, 2×CH₂-cyclopropyl), 0.85 (t, 3H, CH₃ n-propyl), 1.44 (s, 6H, 2×CH₃), 1.46 (m, 2H, CH₂ n-propyl), 2.10-2.30 (m, 4H, 2×CH₂), 2.49-2.66 (m, 5H, 1×CH₂ n-propyl, 1×CH₂, 1×CH), 2.78 and 3.06 (2×m, 2H, CH₂), 401 (t, 1H, CH), 5.89 (bb, 4H, NH³⁺, OH), 703-8.41 (m, 15H, CH═CH and CH-arom.).

The salt of montelukast with n-propylamine was crystallized in an analogous way

from acetonitrile (1 g dissolved under boiling in 40 ml of solvent, yield 64%)
from acetone (1 g dissolved under boiling in 10 ml of solvent, yield 51%)
from ethyl acetate (1 g dissolved under boiling in 40 ml of solvent, yield 63%)
from ethanol (1 g dissolved at the temperature of 55° C. in 10 ml of solvent, yield 42%)
from isopropyl alcohol (1 g dissolved at the temperature of 55° C. in 10 ml of solvent, yield 69%).

### Example 5

### Montelukast Sodium—Amorphous

To 2.11 g of the crystalline salt of montelukast with propylamine, obtained in accordance with Example 3, 15 ml of toluene were added, the suspension was stirred for 20 minutes, then sodium tert-butoxide (0.34 g) and active charcoal was added and the suspension was further stirred at the temperature of approx. 35° C. for 45 minutes. Then, filtration was performed and the clear yellow coloured filtrate was injected into 35 ml of intensively stirred heptane with a syringe. The obtained suspension was stirred for another hour and then it was subject to filtration and vacuum drying. 1.55 g of a powder were obtained. Yield 78%; HPLC 99.6%.

Montelukast sodium was obtained analogously from the salt of montelukast with n-propylamine; yield 82%; HPLC 99.6%.

### Example 6

### Decomposition of Montelukast by the Action of Air Oxygen and Sunshine

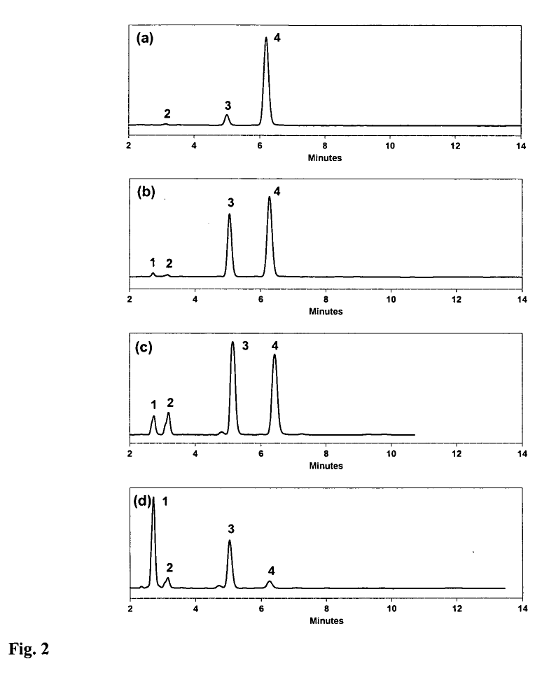

Montelukast (1.0 g), prepared in accordance with Example 5, was dissolved in 100 ml of methanol. The solution in glass apparatus was exposed to the influence of sunshine and air oxygen and samples were take repeatedly (at the times of 40 minutes, 1 day, 4 days and 14 days) (20 μl of the mixture further diluted by methanol to the volume of 1 ml) for HPLC analysis in the isocratic mode. The result of monitoring the changes of the composition is shown in FIG. 2.

### Example 7

### Method of Purification of Montelukast Specifically Contaminated with (Z)-Montelukast

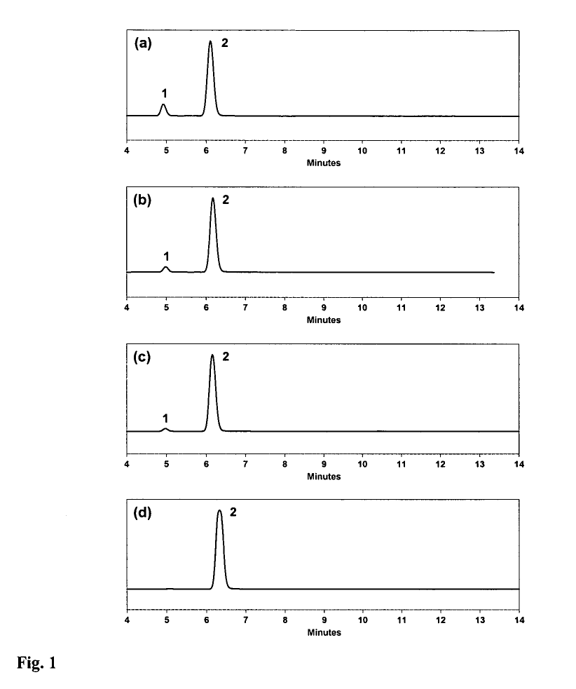

Montelukast (1.0 g), prepared in accordance with Example 5, was dissolved in 100 ml of methanol and this solution was exposed to the influence of sunshine under inert argon atmosphere for 1.5 hours. Subsequently, methanol was evaporated in vacuum and the residue was dissolved in 10 ml of toluene. According to the verification HPCL analysis (isocratic mode) the solution contained approximately 11% of the (Z)-isomer of montelukast; montelukast was the rest up to 100%. This mixture was refluxed in light-insulated atmosphere and under inert atmosphere for 3 hours. Samples were taken in the course of the procedure (20 μl of the mixture further diluted with methanol to the volume of 1 ml) for HPLC analysis in the isocratic mode. The result of monitoring the changes in the composition is presented in FIG. 1. After cooling the toluene solution was injected to an excess of heptane and the obtained suspension was stirred for 1 hour. The separated product was filtered off and dried in vacuum. 0.86 g of montelukast were obtained, which, in accordance with an HPLC analysis (isocratic mode), contained less than 0.1% of the (Z)-isomer.

### Example 8

### Method of Re-Processing of Montelukast Contaminated by Specific Impurities

All the purifying operations of contaminated montelukast (the chemical purity of the starting raw material in accordance with HPLC was 98.75%, content of (E)-montelukast sulfoxide 0.41%, content of montelukast diastereoisomer I 0.18%, content of montelukast diastereoisomer II 0.20%, content of (Z)-montelukast 0.34%, content of the other impurities 0.12% in total) were performed under inert atmosphere and in apparatuses impermeable for light.

Montelukast sodium contaminated by impurities (20 g) was dissolved in toluene (200 ml), the solution was washed with 0.5 M solution of tartaric acid (100 ml), water (50 ml) and the obtained toluene solution was dried over sodium sulfate. Then, the desiccant was filtered off and 4.5 ml of iso-propylamine and 200 ml of heptane were added to the obtained filtrate. After one hour of stirring another 100 ml of heptane were added to the separated suspension and the stirring was continued for one hour. Then filtration was performed, the cake was washed with 1×50 ml of heptane. After vacuum drying at the laboratory temperature 19.3 g of an off-white powder of the salt of montelukast with iso-propylamine were obtained; yield 88%.

The crude salt of montelukast with iso-propylamine was crystallized from isopropyl alcohol and toluene and a product was obtained with the chemical purity of 99.6% and the content of specific impurities below 0.1% according to an HPLC analysis (isocratic mode). 16.8 g of the crystalline salt of montelukast with iso-propylamine were obtained; yield 87%.

To 16.7 g of the obtained crystalline salt of montelukast with iso-propylamine 120 ml of toluene were added, the suspension was stirred for 20 minutes, then sodium tert-butoxide (2.69 g) was added and the suspension was further stirred at the temperature of 30-35° C. for 45 minutes. Then, filtration was performed and the clear filtrate was added dropwise to 280 ml of intensively stirred heptane. The obtained suspension was stirred for another hour and then subjected to filtration and vacuum drying. 14.88 g of an amorphous powder were obtained. Yield 94%.

The yield of the whole process of purification of contaminated montelukast, comprising both the synthesis and crystallization of the salt of montelukast with iso-propylamine and transformation of this salt to the sodium salt of montelukast, was 72%, the chemical purity in accordance with HPLC (gradient mode) was 99.66%, contents of individual impurities were below 0.1%.

Analogously, the process was performed with the salt of montelukast with n-propylamine with the total yield of 77%.

### Example 9

### Preparation of the Standard of 1-[[[(1R)-1-[3-[(1Z)-2-(7-chloro-2-quinolinyl)ethenyl]-phenyl]-3-[2-(1-hydroxy-1-methylethyl)phenyl]propyl]thio]methyl]cyclopropane acetic acid

Montelukast (2.0 g), prepared in accordance with Example 5, was dissolved in 200 ml of methanol and this solution was exposed to the influence of sunshine under inert argon atmosphere for 4 days. According to a verification HPLC analysis (isocratic mode) the solution contained approx. 73% of (Z)-isomer of montelukast; the rest up to 100% contained a majority of montelukast and a minority of other decomposition impurities. Finally, the solvent was evaporated in vacuum, methanol was added to the concentrations residue and it was concentrated in vacuum again. A solid foam was generated and after mechanical disintegration a powder was obtained (ca. 1.45 g with the content of approx. 71% of the (Z)-isomer of montelukast). A portion of the obtained crude (Z)-montelukast was subjected to auto-purification separation in the Waters system (description—see the analytic methods). The output was approx. 300 ml of a solution (acetonitrile/water/formic acid) containing the desired (Z)-isomer with the HPLC purity about 98%. The solvent was evaporated in vacuum (bath temperature 45° C.), pH of the residue was adjusted with a saturated solution of sodium bicarbonate to 7 to 8. Further, the residue was extracted with dichloromethane (3×50 ml), the combined dichloromethane phases were washed with water (2×50 ml) and the solvent was evaporated in vacuum. The evaporation residue was dissolved in methanol and the solvent was evaporated in vacuum again. The oily residue was dissolved in a small volume of dichloromethane and concentrated in vacuum into solid foam (60 mg). The resulting chemical purity of the standard of (Z)-montelukast was 96.05% according to HPLC (gradient mode).

¹H NMR (500 MHz, DMSO-D6), δ (ppm): 0.30-0.38 (m, 4H, 2×CH₂-cyclopropyl), 1.38 a 1.39 (s, 6H, 2×CH₃), 2.23 (m, 2H, CH₂), 2.41 (m, 2H, CH₂), 2.65 and 2.91 (2×m, 2H, CH₂), 3.81 (t, 1H, CH), 6.83 and 7.05 (2×d, 2H, J=12.5 Hz, CH═CH), 6.95-8.10 (13H, CH-arom.).

¹³C NMR (500 MHz, DMSO-D6), δ (ppm): 11.8; 12.0; 16.7; 31.5; 31.6; 38.5; 39.7; 49.2; 71.5; 122.3; 125.0; 125.1; 125.3; 126.2; 127.0; 127.2; 127.6; 128.1; 128.4; 129.6; 130.2; 130.7; 134.1; 134.9; 135.6; 136.1; 139.6; 143.0; 146.6; 147.8; 157.5; 173.1.

MS: 586,2183 (M+1)⁺.

### Example 10

### Preparation of the Standard of [R-(E)]]-1-[[[1-[3-[2-(7-chloro-2-quinolinyl)ethenyl]-phenyl]-3-[2-(1-hydroxy-1-methylethyl)phenyl]propyl]sulfinyl]methyl]cyclopropane acetic acid

6.0 g of montelukast sodium were dissolved in 100 ml of methanol, subsequently 30 ml of 30% hydrogen peroxide were added. After ca. three hours of stirring methanol was evaporated in vacuum and 3 ml of acetic acid were added to the residue. 50 ml of water were added to the separated suspension and the mixture was stirred for 30 minutes; then, it was filtered, the cake was washed with water, with a toluene-heptane (1:1) mixture and finally with heptane. After vacuum drying at 65° C. 5.5 g of a yellow powder were obtained with the meting temp. of 94-100° C. (93%). A portion of the obtained crude (E)-montelukast sulfoxide was subjected to auto-purification separation in the Waters system (description—see analytic methods), while ca. 250 mg of the analytic standard were obtained.

¹H NMR (500 MHz, DMSO-D₆), δ (ppm): 0.34 (m, 1H); 0.48 (m, 1H); 0.63 (m, 1H); 1.42 (s, 3H); 2.22 (m, 1H); 2.26 (m, 1H); 2.44 (m, 1H); 2.46 (m, 1H); 2.61 (d, 1H, J=13.7 Hz); 2.67 (d, 1H, J=13.7 Hz); 2.76 (m, 1H); 2.97 (m, 1H); 4.05 (dd, 1H, J=11.0 a 4.1 Hz); 7.10 (m, 1H); 7.15 (m, 1H); 7.36 (d, 1H, J=7.7 Hz); 7.38 (d, 1H, J=7.6 Hz); 7.48 (t, 1H, J=7.7 Hz); 7.54 (d, 1H, J=16.4 Hz); 7.61 (dd, 1H, J=8.7 a 2.1 Hz); 7.73 (d, 1H, J=7.8 Hz); 7.76 (s, 1H); 7.91 (d, 1H, J=16.4 Hz); 7.96 (d, 1H, J=8.6 Hz); 8.03 (d, 1H, J=8.8 Hz); 8.04 (d, 1H, J=2 Hz); 8.45 (d, 2H, J=8.6 Hz).

¹³C NMR (500 MHz, DMSO-D₆), δ (ppm): 10.8; 12.3; 13.9; 30.9; 31.3; 31.6; 40.1; 56.9; 66.4; 71.6; 120.3; 125.3; 125.6; 126.4; 126.8; 127.0; 127.9; 128.3; 129.3; 129.4; 129.8; 131.0; 134.5; 135.2; 136.0; 136.4; 136.9; 139.6; 146.7; 147.5; 156.5; 172.6.

MS: 602,2125 (M+1)⁺.

### Example 11

### Preparation of 1-[[[(1R)-1-[3-[(1E)-2-(7-chloro-2-quinolinyl)ethenyl]-phenyl]-3-[2-(1-methylethenyl)phenyl]propyl]thio]methyl]cyclopropane acetic acid

4.0 g of montelukast acid were dissolved in 250 ml of toluene, 0.1 ml of methane sulfonyl chloride and 1.5 g of para-toluenesulfonic acid monohydrate were added. The mixture was refluxed under the conditions of azeotropic distillation for approximately 15 hours. Then, the reaction mixture was washed with water, with a 5% solution of sodium bicarbonate, 0.5 M solution of L-tartaric acid and finally with water. The toluene layer was dried over sodium sulfate and concentrated in vacuum after filtration of the desiccant. To the obtained honey-like evaporation residue 15 ml of toluene were added while a yellow crystalline product was separated after a few moments (2.03 g after drying, chemical purity in accordance with HPLC 96.8%).

¹H NMR (500 MHz, DMSO-D₆, 80° C.), δ (ppm): 0.35-0.49 (m, 4H), 1.92 (s, 3H), 2.09-2.18 (m, 2H), 2.31 (m, H), 2.52 (d, 1H), 2.58 (m, 1H), 2.58 (d, 1H), 2.70 (m, 1H), 3.93 (dd, 1H), 4.74 (d, 1H), 5.10 (t, 1H), 7.04 (bd, 1H), 7.12 (m, 1H), 7.17 (m, 2H), 7.40 (t, 1H), 7.45 (d, 1H, J=16.3 Hz), 7.55 (dd, 1H), 7.60 (d, 1H), 7.63 (bd, 1H), 7.65 (s, 1H), 7.87 (d, 1H, J=16.3 Hz), 7.89 (d, 1H), 7.97 (d, 1H), 8.02 (d, 1H), 8.37 (d, 1H).

¹³C NMR (500 MHz, DMSO-D₆, 80° C.), δ (ppm): 11.4; 11.6; 16.3; 24.0; 30.2; 37.7; 38.5; 39.3; 48.9; 114.3; 119.8; 125.2; 125.4; 126.3; 126.4; 126.5; 127.3; 127.6; 127.9; 128.4; 128.5; 129.2; 134.1; 135.1; 135.8; 136.3; 137.3; 142.7; 143.1; 144.6; 147.2; 156.2; 172.1.

MS: 568,2074 (M+1)⁺.

### Example 12

### Preparation of the Standards of 2-[(R)-1-[[[1-[3-[2-(7-chloro-2-quinolinyl)-(S)-1-({[1-(carboxymethyl)cyclopropyl]methyl}thio)ethyl]phenyl]-3-[2-(1-hydroxy-1-methylethyl)phenyl]-propyl]thio]methyl]cyclopropane]acetic acid and 2-[(R)-1-[[[1-[3-[2-(7-chloro-2-quinolinyl)-(R)-1-({[1-(carboxymethyl)cyclopropyl]-methyl}thio)ethyl]phenyl]-3-[2-(1-hydroxy-1-methyl-ethyl)phenyl]propyl]thio]methyl]cyclopropane]acetic acid

To [1-(mercaptomethyl)cyclopropyl]acetic acid (0.7 g) toluene (20 ml), sodium tert-butoxide (0.85 g) and a solution of 2.6 g of PEG-600 in 3 ml of toluene were added under argon atmosphere. Then, a solution of montelukast sodium (2.72 g) in 15 ml of tetrahydrofuran was added dropwise to the stirred mixture. The obtained mixture was stirred at the laboratory temperature and under argon atmosphere for 30 days. Then, toluene (100 ml) was added and 45 ml of the liquid was removed by vacuum distillation. The residue was washed with a solution of tartaric acid and water. The organic phase was dried over sodium sulfate and concentrated to the volume of 30 ml after filtration of the desiccant. To the concentrated residue 3 ml of acetonitrile, 0.5 ml of isopropylamine and gradually 30 ml of heptane were added. The separated suspension of the salt of montelukast with isopropylamine was filtered off and the filtered mother liquor was concentrated in vacuum. 0.4 g of an oily product were obtained, which, according to HPLC, contained 70% of a mixture of montelukast diastereoisomers I and II. The standards of both the diastereoisomers in the form of free acids were obtained in the quantities of approx. 80 mg with the use of the Waters auto-purification system (description—see analytic methods).

### Montelukast Diastereoisomer I:

¹H NMR (500 MHz, CDCl₃), δ (ppm): 0.38 (m, 2H); 0.41-0.48 (m, 4H); 0.56 (m, 2H); 1.60 (s, 3H); 1.61 (s, 3H); 2.00 (d, 1H); 2.40 (d, 1H); 2.16 (q, 2H); 2.33 (d, 1H); 2.43 (d, 1H); 2.35 (d, 1H); 2.55 (d, 1H); 2.37 (d, 1H); 2.49 (d, 1H); 2.97 (q, 2H); 3.37 (dd, 1H); 3.62 (dd, 1H); 3.93 (t, 1H); 4.29 (dd, 1H); 7.11 (m, 1H); 7.17 (m, 2H); 7.23 (m, 1H); 7.24 (m, 1H); 7.28 (m, 1H); 7.30 (m, 1H); 7.38 (d, 1H); 7.4 (dd, 1H); 7.70 (d, 1H); 8.08 (d, 1H); 8.11 (d, 1H).

¹³C NMR (500 MHz, CDCl₃), δ (ppm): 12.2; 12.5; 12.6; 17.1; 17.7; 31.6; 31.8; 32.4; 38.1; 39.2; 39.3; 40.0; 41.2; 45.4; 50.7; 51.2; 123.2; 125.4; 125.5; 126.1; 126.2; 126.8; 127.3; 127.5; 128.2; 128.8; 129.4; 131.8; 135.9; 139.7; 140.7; 142.3; 142.6; 144.6; 147.2; 161.0; 175.7; 175.9.

MS: 432,2586 (M+1)⁺.

### Montelukast Diastereoisomer II:

¹H NMR (500 MHz, CDCl₃), δ (ppm): 0.36-0.62 (m, 8H); 1.56 (s, 3H); 1.57 (s, 3H); 1.98 (m, 1H); 2.13 (m, 1H); 2.00 (m, 1H); 2.46 (m, 1H); 2.20 (d, 1H); 2.65 (d, 1H); 2.36 (d, 1H); 2.48 (d, 1H); 2.77 (m, 1H); 2.96 (m, 1H); 3.29 (dd, 1H); 3.68 (dd, 1H); 3.95 (t, 1H) 4.36 (dd, 1H); 7.09 (m, 1H); 7.10 (m, 1H); 7.16 (dd, 1H); 7.18 (dd, 1H); 7.27 (m, 1H); 7.28 (m, 1H); 7.30 (m, 1H); 7.35 (m, 1H); 7.36 (m, 1H); 7.40 (dd, 1H); 7.61 (d, 1H); 8.02 (d, 1H); 8.09 (d, 1H).

¹³C NMR (500 MHz, CDCl₃), δ (ppm): 12.1; 12.3; 12.5; 12.8; 16.6; 17.0; 31.6; 31.7; 32.1; 38.8; 39.1; 40.0; 40.5; 45.7; 50.3; 51.0; 74.3; 123.3; 125.3; 125.4; 125.6; 125.9; 126.8; 127.1; 127.2; 127.4; 128.2; 129.1; 131.7; 135.9; 136.7; 140.4; 142.4; 142.7; 144.8; 147.3; 161.0; 175.9; 176.2.

MS: 432,2591 (M+1)⁺.

### Example 13

### Preparation of the Standards of 2-[2-(3-{3-[2-(7-chloroquinolin-2-yl)vinyl]phenyl}-allyl)phenyl]-propan-2-ol and 7-chloro-2-{2-[3-(1,1-dimethyl-1,3,4,5-tetrahydrobenzo[c]-oxepin-3-yl)phenyl]vinyl}quinoline

24.9 g of a paste of montelukast mesylate with acetonitrile, containing ca. 83% by weight of the mesylate were put into a vacuum drier and dried at the temperature of 95° C. and vacuum of 10 mbar for 4 hours. 20.8 g of a yellow powder were obtained, which contained a mixture of montelukast cyclizate and montelukast eliminate in the approximate proportion of 2:1. A portion of the obtained mixture was mixed with ca. 100 ml of chloroform. A certain portion of the mass remained undissolved. Filtration and drying was performed. 2.9 g of a yellow powder were obtained, which contained crude montelukast eliminate in the form of a salt with methane sulfonic acid. The crude product was washed with ether (100 ml) and a mixture of 200 ml of chloroform and 50 ml of ether. 1.67 ml of the standard of montelukast eliminate were obtained in the form of its salt with methane sulfonic acid.

Salt of Montelukast Eliminate with Methane Sulfonic Acid:

¹H NMR (500 MHz, DMF-D₆, 30° C.), δ (ppm): 1.67 (2×s, 2×3H), 4.09 (d, 1H, J=6.6 Hz), 6.62 (d, 1H, J=15.8 Hz), 6.75 (dt, 1H, J=15.8 and 6.6 Hz), 7.21 (td, 1H, J=7.5 and 1.4 Hz), 7.25 (td, 1H, J=7.3 and 1.4 Hz), 7.35 (dd, 1H, J=7.4 and 1.4 Hz), 7.47 (t, 1H, J=7.6), 7.52 (d, 1H, J=7.6 Hz), 7.52 (dd, 1H, J=7.6 and 1.1 Hz), 7.71 (d, 1H, J=7.5 Hz), 7.78 (dd, 1H, J=8.8 and 2.0 Hz), 7.94 (s, 1H), 8.20 (d, 1H, 14.9 Hz), 8.25 (m, 1H), 8.25 (m, 1H), 8.28 (d, 1H, 8.8 Hz), 8.77 (d, 1H, J=8.8 Hz).

MS: 440,1777 (M+1)⁺.

The fraction of the initial mixture soluble in chloroform (2:1) was enriched with montelukast cyclizate. This solution was filtered through a layer of silica gel while 5 fractions were withdrawn. After evaluation of analyses of the withdrawn fractions (HPLC and TLC) the fraction containing the product was concentrated. 50 ml of ether were added to the oily distillation residue. The obtained yellow suspension was filtered off; the filtration cake was washed with ether and dried. 0.36 g of a light yellow powder were obtained, which contained montelukast cyclizate in the form of its salt with methane sulfonic acid.

Salt of Montelukast Cyclizate with Methane Sulfonic Acid:

¹H NMR (500 MHz, DMSO-D₆), δ (ppm): 1.54 (s, 3H, CH₃), 1.62 (s, 3H, CH₃), 1.93 and 2.17 (m, 2H CH₂), 2.42 (s, 3H, CH₃—S), 2.63 and 3.43 (m, 2H, CH₂), 4.63 (dd, 1H, CH), 7.17-7.27 (m, 4H), 7.42 (d, 2H), 7.57 (d, 2H), 7.61 (m, 1H), 7.75 (m, 1H), 7.79 (s, 1H), 8.08-8.15 (m, 4H), 8.70 (d, 1H).

MS: 440,1785 (M+1)⁺.

ANALYTIC METHODS (A, B): The process of the preparation of montelukast, the composition of the reaction mixtures exposed to light and oxygen load, as well as the quality of the target substance including its salts with amines and of isolated standards of impurities were controlled by means of high performance liquid chromatography (HPLC). An isocratic, as well as gradient HPLC methods have been developed (A). The standards of specific impurities of montelukast were obtained by separations with the use of the Waters auto-purification system (B).

### A High Performance Liquid Chromatography (HPLC)

### Isocratic Method

HPLC chromatograms were measured with the EliteLachrom device of Hitachi. For the analyses a column filled with the stationary phase of RP-18e was used, column temperature 20° C. As the mobile phase a mixture of acetonitrile (80%) and a 0.1 M aqueous solution of ammonium formate, treated with formic acid to pH 3.6 (20%), was used. The measurements were performed in the isocratic mode with the mobile phase flow rate of 1.5 ml/min. Spectrophotometric detection at the wavelength of 234 nm was used. For the preparation of the samples to be analyzed methanol was used as the solvent, 10-20 μl of the prepared solution were used for the injection.

### Gradient Method (Chemical Purity)

Instrumentation: Alliance HPLC, PDA detector
Column: Purospher STAR RP8e, 250×4.0 mm, 5 um (Merck)
Mobile phase: A: phosphate buffer 0.01 M (1.4 g) KH₂PO₄ is dissolved in 1 l MQ of water,
pH of the solution is adjusted to 2.2±0.05 with phosphoric acid
B: Acetonitrile
Elution: Gradient type

| Time (min.) | Flow (ml/min.) | % A | % B |
|---|---|---|---|
| 0 | 0.8 | 60 | 40 |
| 20 | 0.8 | 15 | 85 |
| 25 | 0.8 | 15 | 85 |
| 30 | 0.8 | 60 | 40 |
| 35 | 0.8 | 60 | 40 |

Sample solvent: methanol
Detection: spectrophotometric 238 nm
Injection: 10 μl
Auto-sampler temperature: 10° C.
Column temperature: 15° C.
Analysis time:
Tested solution: 1 mg/1 ml

TABLE 1
Sequence of peaks in the gradient HPLC method for known
montelukast impurities, including montelukast, with the
specification of the relative retention times (RRT).

| Seq. No. | Identification | RRT |
|---|---|---|
| 1. | (Z)-montelukast sulfoxide | 0.59 |
| 2. | (E)-montelukast sulfoxide | 0.67 |
| 3. | montelukast alcohol | 0.75 |
| 4. | montelukast diastereoisomer I | 0.87 |
| 5. | montelukast diastereoisomer II | 0.89 |
| 6. | (Z)-montelukast | 0.95 |
| 7. | montelukast | 1.0 |
| 8. | montelukast eliminate | 1.12 |
| 9. | montelukast dehydrated | 1.29 |
| 10. | montelukast cyclizate | 1.38 |

### B Auto-Purification System for Separation of Substance Mixtures

A Waters auto-purification system with an SQ detector, XBridge Prep OBD C18 column, 100×19 mm, 5 μm (Waters) mixture of two mobile phases A (0.1% formic acid in water) and B (acetonitrile) was used. For separation of specific impurities of montelukast ((V-A), (IV-A) (XIIIa-A) and (XIIIb-A)) the following three purification methods with gradient elution were used.

### Purification Method of the Impurity (Z)-Montelukast (V-A)

Elution: gradient-type

| Time (min) | A (%) | B (%) |
|---|---|---|
| 0 | 50 | 50 |
| 1 | 50 | 50 |
| 8 | 20 | 80 |
| 11 | 20 | 80 |
| 12 | 50 | 50 |
| 15 | 50 | 50 |

Flow: 20 ml/min. Detection: MS, ESI+ ionization, collection of full m/z spectra 500-700 (primary ion of (Z)-montelukast—m/z+586) UV—238 nm, Column temperature: 25° C. Sample temperature: 25° C. Injection: 500 μl. Preparation time: 15 min. Sample preparation: 0.5 g is dissolved in 10 ml of methanol. Collection of fractions: ESI+, MIT—3000000, peak start—MIT only, peak end—MIT only, Fraction trigger: mixed—mass A (+586) not mass B (+584).

### Purification Method of the Impurity (E)-Montelukast Sulfoxide (IV-A)

Elution: gradient-type

| Time (min) | A (%) | B (%) |
|---|---|---|
| 0 | 60 | 40 |
| 10 | 60 | 40 |
| 15 | 40 | 60 |
| 16 | 10 | 90 |
| 17 | 10 | 90 |
| 18 | 60 | 40 |
| 20 | 60 | 40 |

Flow: 20 ml/min. Detection: MS, ESI+ ionization, collection of full m/z spectra 500-700 (primary ion of (E)-montelukast sulfoxide—m/z+602) UV—238 nm. Column temperature: 25° C. Sample temperature: 25° C. Injection: 500 μl. Preparation time: 20 min. Sample preparation: 2.5 g are dissolved in 50 ml of methanol. Collection of fractions: UV: MIT—1000000, peak start—MIT only, peak end—MIT only. Fraction trigger—UV 238 nm

### Purification Method of Diastereoisomers (XIIIa-A), (XIIIb-A)

Elution: gradient-type

| Time (min) | A (%) | B (%) |
|---|---|---|
| 0 | 60 | 40 |
| 2 | 60 | 40 |
| 15 | 40 | 60 |
| 25 | 40 | 60 |
| 26 | 60 | 40 |
| 30 | 60 | 40 |

Flow: 20 ml/min. Detection: MS, ESI+ ionization, collection of full m/z spectra 500-1000 (primary ion of the diastereoisomers (XIIIa-A), (XIIIb-A)—m/z+732) UV—238 nm. Column temperature: 25° C. Sample temperature: 25° C. Injection: 500 μl. Preparation time: 30 min. Sample preparation: 0.5 g of the sample is dissolved in 10 ml of methanol. Collection of fractions: UV: MIT—200000, peak start—MIT only, peak end—MIT only. Fraction trigger: UV 238 nm.

1-41. (canceled)

42. An isolated specific impurity of montelukast, selected from the group including 2-[(R)-1-[[[1-[3-[2-(7-chloro-2-quinolinyl)-(S)-1-({[1-(carboxymethyl)cyclopropyl]methyl}thio)ethyl]-phenyl]-3-[2-(1-hydroxy-1-methylethyl)phenyl]propyl]thio]methyl]cyclopropane]-acetic acid of formula XIIIa: 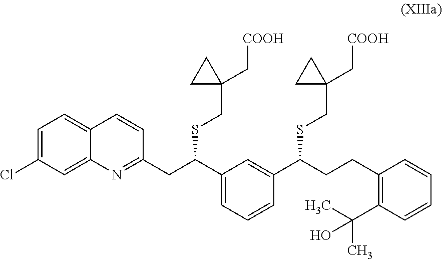
and 2-[(R)-1-[[[1-[3-[2-(7-chloro-2-quinolinyl)-(R)-1-({[1-(carboxymethyl)cyclopropyl]methyl}thio)ethyl]-phenyl]-3-[2-(1-hydroxy-1-methylethyl)phenyl]propyl]thio]methyl]cyclopropane]-acetic acid of formula XIIIb: 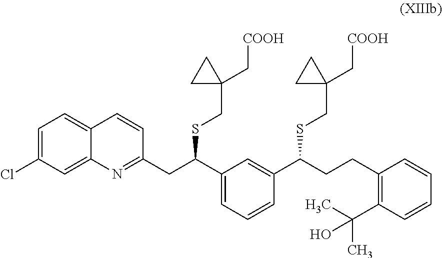
and their alkali metal salts, characterized by chemical purity of more than 50% for use in setting the analytic methods designed for quality control of montelukast.

43. The isolated specific impurity of montelukast as defined in claim 42, characterized by a chemical purity of 95% or more.

44. A method for the isolation of the montelukast impurity as defined in claim 42 from mixtures containing montelukast, wherein the content of this impurity is increased by the effect of a salt of [1-(mercaptomethyl)-cyclopropyl]acetic acid with an alkaline metal on a solution of montelukast or its salt, followed by use of the auto-purification technique with the possibility of combined UV and weight detection of the separated constituents.

45. A method for controlling the production process of montelukast, said method comprising the step of using the isolated impurity according to claim 42 as a reference standard in an analytical method.

46. The method according to claim 45, wherein said analytical method is an HPLC method.

47. A method for preparing pharmaceutical products, said method comprising the step of using the isolated impurity according to claim 42 as a reference standard for the quality assessment of montelukast sodium.

48. A method for controlling the production process of montelukast, said method comprising the step of using the isolated impurity according to claim 43 as a reference standard in an analytical method.

49. The method according to claim 48, wherein said analytical method is an HPLC method.

50. A method for preparing pharmaceutical products, said method comprising the step of using the isolated impurity according to claim 43 as a reference standard for the quality assessment of montelukast sodium.
